(19) United States
(12) Patent Application Publication
(10) Pub. No.: US 2009/0088633 A1
(43) Pub. Date: Apr. 2, 2009

(54) Method for the combined image display of a catheter inserted into the heart area of a patient with electrophysiological cardiological data

(75) Inventors: Andreas Meyer, Bubenreuth (DE); Norbert Rahn, Forchheim (DE); Dietrich Till, Forchheim (DE)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Aktiengesellschaft

(21) Appl. No.: 12/284,597

(22) Filed: Sep. 23, 2008

(30) Foreign Application Priority Data
Sep. 28, 2007 (DE) 10 2007 046 938.3

(51) Int. Cl. A61B 6/00

(52) U.S. Cl. 600/424; 378/4

(57) ABSTRACT

The invention relates to a method for the combined three-dimensional image display of a catheter. The catheter is inserted into the heart area of a patient with electrophysiological data, as part of a cardiological investigation or treatment. A current position of the catheter is localized with fluoroscopy-aided. The current position of the catheter and the electrophysiological data of the patient are blended into a three-dimensional volumetric image of a structure of the heart for the combined three-dimensional image display.

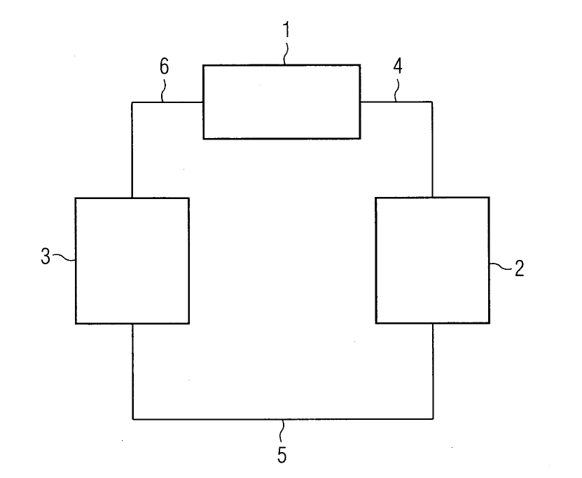

### CROSS REFERENCE TO RELATED APPLICATIONS

This application claims priority of German application No. 10 2007 046 938.3 filed Sep. 28, 2007, which is incorporated by reference herein in its entirety.

### FIELD OF THE INVENTION

The invention relates to a method for the combined three-dimensional (3D) image display of both a medical instrument inserted into the heart area of a patient, especially a catheter, and electrophysiological data, including also the position of the ablation and mapping points, as part of a cardiological investigation or treatment.

### BACKGROUND OF THE INVENTION

In electrophysiology, cardiac disrythmia (arrhythmia) is increasingly being treated by catheter ablation techniques. For this purpose, it is essential that the examining physician can determine the position of the catheter within the heart in order to be able to obtain a precise orientation. The physician must be able to verify, at any time during the investigation, the position of the catheter and the points in the heart at which ablation is being carried out. The minimally invasive intervention of catheter ablation is applied as tissue sclerosing using several ablation sequences. It is, however, frequently the case that after setting a linear or circular lesion, gaps remain which have to be subsequently closed for successful therapy.

The position of the catheter in the heart, which is required for the electrophysiological investigation (EPU), can be most simply shown by using a monoplanar or biplanar fluoroscopy system. The disadvantage of this method is, however, in the two-dimensional nature of the X-ray projection image. This means that the third dimension of the space is lost and also cannot be completely compensated for by biplanar fluoroscopy, because the three-dimensional position of the catheter in the heart can be assembled by the examiner from the two projection images only by imagination. A further disadvantage of this method lies in the fact that the hyperemic structure of the heart is not, or only very poorly, shown in the image, so that the examiner has to rely completely on changes in the shape of the catheter or on the effects of movement of the cardiac wall on the movement of the catheter in order to obtain visual information on the current position of the catheter.

For this reason, three-dimensional “mapping” systems based either on the principal of measurement of magnetic fields or on the principal of impedance measurement have been available on the market for some time. These modern mapping methods include electro-anatomical mapping (Carto), non-contact mapping (Ensite), multipolar basket mapping and the three-dimensional localization of intracardial electrodes (Localisa). With the aid of these systems, the catheter position can be displayed in three dimensions and a mapping (i.e. the cartographic representation of the electrical excitation conduction in the heart) of the structures of the heart can be produced. Furthermore, these systems can provide a color-coded display of the propagation of the electrical excitation in the heart or the electrical voltage as a function of the location in space and can also plot the ablation points.

The known mapping systems are briefly explained in the following.

The electro-anatomical mapping with the Carto system is based on electromagnetic principles. Three different alternating magnetic fields of low intensity are built up under the examining table. By means of integral electromagnetic sensors at the point of the catheter, it is possible to measure the voltage changes within the magnetic field induced by catheter movements and to calculate the position of the mapping catheter (to within an accuracy of approximately 1 mm) at any timepoint with the aid of mathematical algorithms. By recording the anatomy of a heart cavity by scanning the endocardial contour with the mapping catheter and the simultaneous registration of electrical signals, an electro-anatomical map is produced.

The non-contact mapping using the Ensite system is based on different principles. This is a simultaneous mapping method with the simultaneous recording of more than 3000 “virtual” electrograms. In this case, the multielectrode catheter used has no direct contact with the cardiac wall. This is enabled by the registration of voltage changes during the endocardial depolarization. A fine copper wire mesh with a total of 64 poles, mounted on an 8.5 French F balloon detects these voltage changes, which by means of a complicated mathematical process are displayed on a computer workstation as unipolar electrograms with color-coded three-dimensional excitation fronts.

Multipolar basket mapping represents a further simultaneous mapping method and is used primarily for the diagnosis of atrial arrhythmia. By a basket-shaped clamping of very resilient self-expanding electrode splines of nickel titanium, which lie against the endocardium, up to 56 bipolar electrograms can be recorded by means of 64 platinium-irridium electrodes. The three-dimensional localization method of intracardial electrodes (Localisa) enables a three-dimensional determination of the position of conventional electrodes by measuring the impedance of a weak electrical current (after external cutaneous application).

However, electrophysiological investigation systems of this kind require additional expenditure going beyond the fluoroscopic system, which is present in any case. This means that during an electrophysiological procedure additional financial expenditure for the special catheters or other ongoing consumables occur in addition to the longer investigation duration.

### SUMMARY OF THE INVENTION

The object of this invention is to provide a method by means of which electrophysiological data can be blended into the three-dimensional volumetric image of the heart and the precise three-dimensional spatial orientation of a catheter in the heart can be determined at the same time using the existing fluoroscopic system, as part of a cardiological investigation or treatment.

To achieve this object, a method with the following steps is provided.
a) Generating a three-dimensional volumetric image of the structure of the heart,
b) Registering the three-dimensional volumetric image relative to the coordinates of a biplanar system,
c) Determining the three-dimensional spatial orientation of a catheter by means of the feedback projection of the catheter from two X-ray projections of a biplanar system and generating electrophysiological data
d) Blending the current catheter position and the electrophysiological data into the three-dimensional volumetric image of the structure of the heart.

Therefore, this method is based on two important components of prior art, as follows.
a) Fluoroscopy-aided catheter localization and
b) the registered three-dimensional representation of the structure of the heart by means of fluoroscopy

Re a)

The fluoroscopy-aided catheter localization is based on the feedback projection of the catheter from two X-ray projections of a biplanar system. The biplanar system is an X-ray system which enables two X-ray images to be recorded from two different directions, for example by means of two C-arms. In this way, it is possible to identify the catheter by means of the two-dimensional fluoroscopic images and to then calculate a feedback projection line using the respective projection matrix of the particular two-dimensional fluoroscopic image, with the spatial position of the catheter being determined by means of the feedback projection lines and ideally lying in the intersection point of the two projecting lines. Due to design conditions which mean that the radiation source and the radiation detector do not take up precisely the same position relative to each other at the particular positions in which the fluoroscopic images are recorded, it is frequently the case that the calculated feedback projection lines do not intersect. In such a case, it is advantageous to mathematically determine the position of the shape so that using the non-intersecting feedback projection lines a position is calculated that is close to the positions of the catheter point identified in the two-dimensional fluoroscopic images. For this purpose, any point in the given volume that changes its position during the optimization process until it is nearest to the identified position of the catheter point in the two-dimensional images can, for example, be used. As an alternative, it is also possible to determine the center of the imaginary feedback projection lines at the point of their minimum distance as a calculated position. The location, i.e. the spatial position and spatial orientation of the catheter is determined in three dimensions. This is possible because the three-dimensional volumetric image and both two-dimensional fluoroscopic images are registered relative to each other, i.e. their systems of coordinates are correlated relative to each other by a transformation matrix. A method of this kind is, for example, known from document DE 102 10 647 A1.

Re b)

The registered three-dimensional representation of the structure of the heart for fluoroscopy can be achieved in different ways. On the one hand, the three-dimensional record of the heart can be generated before the procedure, i.e. pre-procedurally, on a different scanner (e.g. CT or MR=magnetic resonance) and then registered relative to the X-ray system at the start of the procedure, with it being possible to use a wide variety of methods, e.g. a two-dimensional/three-dimensional registration or a three-dimensional/three-dimensional registration. Furthermore, the three-dimensional representation can be generated directly on the operating table with the aid of rotation-angiography and subsequent reconstruction. This is known as the “Cardiac DynaCT” method. With this method, the registration is provided intrinsically on the condition that the patient does not move during the procedure following the reconstruction.

A two-dimensional/three-dimensional registration is present if the two-dimensional X-ray fluoroscopy images are combined with a three-dimensional volumetric image (CT, MR, Cardiac DynaCT). If both CT or MRI image data recorded pre-procedurally or pre-operatively and also X-ray rotation-angiography image data recorded intra-procedurally or intra-operatively is present, a three-dimensional/three-dimensional registration of both recordings can be made. In any case, a two-dimensional/three-dimensional registration is to be carried out if a patient movement, or undetected movement (such as sagging) of the patient table, occurs between the three-dimensional recording and the recording of the two-dimensional fluoroscopic images.

The three-dimensional recording or the three-dimensional volumetric image of the heart can, according to the invention, be a data record made pre-procedurally or pre-operatively. This means that the data record can have been taken at any timepoint before the actual investigation or treatment. Any three-dimensional image data record can be used regardless of the recording modality employed, i.e. for example a CT, an MR or a three-dimensional X-ray angiography data record. All these data records enable an exact reconstruction of the heart so that an anatomically-exact high-resolution display of the heart can be obtained. Alternatively, it is possible to also use a data record in the form of a three-dimensional X-ray angiography data record taken intra-procedurally or intra-operatively. The term “intra-procedural” in this case means that the data record is obtained with an immediate time relationship to the actual investigation or treatment, i.e. when the patient is already on the examining table but the catheter has not yet been inserted but is inserted shortly after taking the three-dimensional image data record.

In this invention, both the current catheter position, which has been established by fluoroscopically-aided catheter localization, and the electrophysiological data of the heart are blended into the three-dimensional volumetric image of the structure of the heart. This blending-in can optionally be triggered by ECG, which means that either for the blending-in the images of the correct heart phase must be chosen for the feedback projection or the ECG triggering X-radiation must be activated in the correct heart phase. The correct heart phase is the heart phase in which the three-dimensional volumetric image was generated. Ideally, the heart is externally stimulated (pacing) during this intervention in order to achieve a stable heart frequency and thus a predictable triggering of the X-radiation.

Both the acquisition of the image data and the reconstruction of the image data can be controlled, i.e. triggered, by the pacing signal. This can be achieved by the simultaneous recording of various image data during the recording of the pacing signal, with the image data which was recorded in a specific heart phase being afterwards combined and reconstructed to form a three-dimensional image data record.

Pacing is normally possible without difficulty in the field of electrophysiological investigation because it is used in any case for many medical problems.

The electrophysiological data can be obtained by an ECG, for example an intracardial or extracardial ECG. By derivation of the electrical signals in the heart (catheter mapping), the points at which an ablation needs to be carried out can be determined as part of a cardiological investigation or treatment. The use of an intracardial ECG is preferred.

By means of this invention, the electrophysiological measuring station is connected to the three-dimensional catheter localization. It is thus possible to blend in or transfer the electrophysiological data into the three-dimensional volumetric image of the structure of the heart, with it being possible to locate the catheter at the same time.

Electrophysiological mapping systems according to prior art are able to blend in the ablation points, color-coded, into the electro-anatomical map. The system described in the invention is able to blend in this information directly into the three-dimensional volumetric image because in this case only the biplanar three-dimensional position of the catheter determined by X-radiation needs to be linked into the “ablation on” information from the ECG measuring station and can then be blended into the three-dimensional volumetric image as an ablation point. As an option, manual triggering can be used to enable all positions of the catheter point to also be shown marked in the three-dimensional reconstruction, e.g. mapping points. Furthermore, a color-coded blending-in of the electrical signal propagation or the time relationships of the signal propagation into the three-dimensional volumetric image is possible.

With the aid of this invention, it is possible for the examining physician to determine the position of the catheter within the heart, to spatially orient himself precisely and also to know the point in the heart at which he has performed ablations. The invention therefore enables not only the blending-in of the current catheter position into the three-dimensional volumetric image but also the blending-in of electrophysiological data.

It is particularly advantageous for the physician if the combined image display of the structure of the heart with the blended-in catheter can be guided, changed (especially rotated), enlarged or reduced by the user so that in this way he can even more precisely establish the position of the catheter in the heart and thus, for example, very closely determine the proximity to a heart wall. The catheter can be shown colored or flashing to improve visibility.

In addition to the inventive method, this invention relates to a medical investigation and/or treatment device, designed to perform the method of the described type. Particularly preferred in this case is an investigation and/or treatment device which combines an electrophysiological measuring station with a three-dimensional workstation computer on which the catheter localization takes place.

### BRIEF DESCRIPTION OF THE DRAWINGS

The invention is now explained in more detail by means of an exemplary embodiment with reference to the accompanying drawing showing the principle of an inventive medical investigation and/or treatment device.

### DETAILED DESCRIPTION OF THE INVENTION

FIGURE shows an X-ray system 1. The X-ray system allows both the acquisition of two-dimensional fluoroscopy images and also the acquisition of a series of two-dimensional images during a rotation. The X-ray system 1 is connected to a three-dimensional workstation computer 2. In this way, an image transmission 4 from the X-ray system 1 to the three-dimensional workstation computer 2 can take place. The three-dimensional workstation computer can preferably read in the three-dimensional volumetric image, register the position of a pre-procedural or intra-procedural three-dimensional volumetric image relative to the coordinates of the X-ray system and register the spatial orientation of the catheter, thereby blending the spatial position and spatial orientation of the catheter into the three-dimensional volumetric image of the structure of the heart. An ECG is obtained at the same time, with the three-dimensional workstation computer 2 being connected by a coupling 5 to an electrophysiological workstation system 3. The information from the electrophysiological workstation system 3 is then processed in the three-dimensional workstation computer 2, i.e. the assignment of the electrophysiological data to the catheter position and the coupling of the electrophysiological data to the catheter position preferably takes place. Thus, for example, the ablation points can be blended-in, color-coded, into the three-dimensional volumetric image. Furthermore, the electrophysiological workstation system 3 is connected to the X-ray system 1, so that the recording of the X-ray images can be ECG-triggered, with the ECG trigger information 6 being sent from the electrophysiological workstation system 3 to the X-ray system 1. With this investigation/treatment device, a color-coded blending-in of the electrical signal propagation or voltage mapping in the three-dimensional volumetric image is also possible.

This invention thus enables a combined three-dimensional image display of electrophysiological data with the catheter localization.

1.-10. (canceled)

11. A method for a three-dimensional image display comprising electrophysiological data of a heart of a patient and a catheter inserted into the heart of the patient, comprising:
generating a three-dimensional volumetric image of a structure of the heart;
registering the three-dimensional volumetric image relative to a coordinate of a biplanar X-ray system;
recording two X-ray projections of the catheter in two different directions by the biplanar X-ray system;
determining a current position of the catheter by feedback projecting the two X-ray projections of the catheter;
generating the electrophysiological data; and
blending-in the current position of the catheter and the electrophysiological data into the three-dimensional volumetric image for the three-dimensional image display.

12. The method as claimed in claim 11, wherein the three-dimensional volumetric image of the structure of the heart is recorded pre-procedurally.

13. The method as claimed in claim 11, wherein the three-dimensional volumetric image of the structure of the heart is recorded intra-operatively.

14. The method as claimed in claim 11, wherein the current position of the catheter is blended into the three-dimensional volumetric image via ECG-triggered.

15. The method as claimed in claim 11, wherein the electrophysiological data comprises a position of an ablation or a mapping point of the current position of the catheter in the three-dimensional volumetric image of the structure of the heart.

16. The method as claimed in claim 15, wherein the ablation or the mapping point is color-coded blended into the three-dimensional volumetric image of the structure of the heart.

17. The method as claimed in claim 11, wherein the three-dimensional image display is controlled by a user for rotating, enlarging, or reducing.

18. The method as claimed in claim 11, wherein the current position of the catheter is displayed by a color or flashing in addition to the electrophysiological data.

19. The method as claimed in claim 11, wherein the current position of the catheter is three-dimensional.

20. A medical device, comprising:
a biplanar X-ray system that records two X-ray projections of a catheter to be inserted into a heart of a patient in two different directions;
an electrophysiological measuring device that generates an electrophysiological data of the heart of the patient; and
a computer that:
registers a three-dimensional volumetric image of a structure of the heart relative to a coordinate of the biplanar X-ray system,
determines a current position of the catheter by feedback projecting the two X-ray projections of the catheter,
blends-in the current position of the catheter and the electrophysiological data into the three-dimensional volumetric image, and
displays the three-dimensional volumetric image blended in the current position of the catheter and the electrophysiological data.
